(19) United States
(12) Patent Application Publication
(10) Pub. No.: US 2007/0082859 A1
(43) Pub. Date: Apr. 12, 2007

(54) Formulations of AICA riboside

(76) Inventors: Richard R. Stover, West Cornwall, CT (US); Ross Dixon, La Jolla, CA (US); Dennis T. Mangano, Hillsborough, CA (US); Sharon Wu, San Diego, CA (US); Marvin J. Sack, Overland Park, KS (US)

Correspondence Address: WILSON SONSINI GOODRICH & ROSATI, 650 PAGE MILL ROAD, PALO ALTO, CA, 94304-1050, US

(21) Appl. No.: 11/246,763

(22) Filed: Oct. 7, 2005

(51) Int. Cl. A61K 31/7056

(52) U.S. Cl. 514/43

(57) ABSTRACT

This invention relates to kits, compositions, and formulations comprising AICA riboside and analogs thereof and methods of using the same. The kits may comprise the compositions and/or formulations within a pH range, in a sterilized form. The kits may further comprise containers with a second therapeutic agent. The kits and compositions herein can be used to prevent morbidity/mortality in a patient or to prevent/treat an ischemic condition, a condition regulated by adenosine, or a condition associated with reduced blood flow to a tissue in a patient.

### BACKGROUND

Ischemia is a low oxygen state typically caused by inadequate blood flow to a tissue resulting in reduced oxygen supply to the tissue, or hypoxia. Several types of ischemia exist including myocardial, mesenteric, and cerebral. It is known that the purine nucleoside, adenosine, can exert a protective effect under ischemic conditions. Acadesine or 5-aminoimidazole-4-carboxamide (AICA) riboside, a precursor molecule of nucleotide biosynthesis, can enhance the local endogenous levels of extracellular adenosine during periods of ischemia. It would be useful to find formulations of AICA riboside and/or analogs thereof which can be used to treat/prevent ischemic conditions, conditions regulated by adenosine, effects of reduced blood flow to a tissue or simply prevent morbidity/mortality in a patient.

### SUMMARY OF THE INVENTION

The present invention relates to a buffered solution comprising or consisting essentially of a composition such as AICA riboside or an analog therof (e.g. formula III). In some embodiments, such solutions have pH between 6.5 and 7.5. Preferably the AICA riboside or analog thereof is sterilized, e.g., by lyophilization or non-lyophilization. In some embodiments, the above solution has less than 1% impurity by weight.

In some embodiments, the invention relates to a perfusate solution. Such perfusate solution comprises, consists essentially of, or consists of the buffered solution described above.

The compositions/solutions (including perfusate solution) herein can include a second therapeutic agent. Examples of a second therapeutic agent include, but are not limited to an adenosine deaminase inhibitor, a blood-clotting inhibitor, an anticoagulant, an anti-platelet agent, an anti-hypertensive agent, a cholesterol-lowering drug, a vasodilator, a beta-blocker, an ace-inhibitor, an analgesic, an anti-inflammatory agent, and a diuretic. In some embodiments, the second therapeutic agent is pentostatin.

The present invention also relates to methods of treating or preventing a condition in a patient wherein the method comprises the step of administering to the patient an effective amount of the solution described above.

Examples of condition that can be treated or prevented by the compositions and solutions herein include those selected from the group consisting of a heart attack, a stroke, death (e.g., sudden cardiac death), a myocardial infarction (e.g., a transmural or non-transmural myocardial infarction or an acute myocardial infarction), coronary artery disease, coronary heart disease, an arrhythmia, a cerebrovascular accident, congestive heart failure, a life-threatening dysrhythmia, cardiomyopathy, an ischemic condition (e.g., a transient ischemic attack, an acute ischemic syndrome, acute bowel ischemia, kidney ischemia), angina pectoralis, a vascular condition (e.g., microvascular disease of diabetes mellitus, disseminated intravascular coagulation such as due to bowel ischemia), intermittant claudication of skeletal muscle, migraine headaches, Raynaud's phenomenon, a hepatic injury, a pancreatic injury, shock, or a combination thereof. In some embodiments, the condition is death, a myocardial infarction or a heart attack.

In some embodiments, the patient being administered a composition/solution of the present invention is undergoing surgery. In such embodiment, the compositions/solutions herein can be administered perioperatively. In some embodiments, the patient is undergoing a cardiac surgery; while in other embodiments, the patient is undergoing a non-cardiac surgery.

Examples of cardiac surgeries include those selected from the group consisting of coronary artery bypass grafting (CABG), percutaneous transluminal coronary angioplasty (PTCA), laser angioplasty, cerebral angioplasty, an atherectomy, an intravascular stent procedure, carotid endarterectomy, heart transplant, implantation of artificial heart devices and defibrillators, valve replacement or repair, and congenital surgery. In preferred embodiments, the compositions/solutions herein are administered to a patient undergoing a CABG surgery.

Examples of non-cardiac surgeries include those selected from the group consisting of non-heart organ transplant, small and large bowel resection, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, and liposuction, or a combination thereof.

In some embodiments, the compositions/solutions herein are administered percutaneously or preferably i.v. injection or via a perfusate solution in an effective amount.

An effective amount of the compositions herein include an amount that is between 0.001 mg/kg/min to 20 mg/kg/min, or more preferably about 0.1 mg/kg/min. At such rates, the compositions/solutions herein can be administered for at least 1 hour, or more preferably for about 7 hours.

The methods herein can prevent any of the conditions herein, but preferably death, stroke, or a myocardial infarction in a patient undergoing CABG surgery.

The present invention also contemplates kits comprising a first container which comprises, consists essentially of, or consists of a solution of the present invention and instructions for use thereof in preventing a condition in a patient.

### BRIEF DESCRIPTION OF THE FIGURES

FIG. 1 illustrates a representative kit of the invention that may include a composition comprising AICA riboside suitable for use in preparing an intravenous solution and/or perfusate solution, and instructions for use.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The term “co-administered” as used herein refers to administration of two or more agents as part of the same treatment plan, whether or not simultaneous in time or not. Thus, agents that are co-administered can be co-formulated or independently formulated. Preferably, two agents are co-administered such that their biological activity overlaps in time.

The term “patient” is preferably a mammal, and more preferably a human.

The term “treat”, “treating” or “treatment” as used herein refers to prevention as well as amelioration or reduction of a symptom or a condition affecting an organism.

Overviews

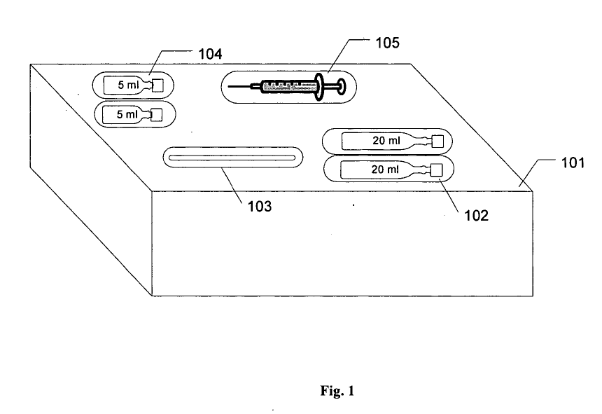

FIG. 1 is an illustration of a kit 101 for treatment and/or prevention of an ischemic condition, or more preferably a cardiovascular ischemic condition, or more preferably prevention of a myocardial infarction.

Kit 101 includes a first container 102 comprising sterilizing AICA riboside (or analog, metabolite, prodrug, or salt thereof). In some embodiments, such sterilized AICA riboside is lyophilized. In other embodiments, such sterilized AICA riboside is sterilized but non-lyophilized.

Such sterilized AICA riboside is preferably in a buffered solution. A buffered solution can have a stable pH range of 6-8 or more preferably 6.5-7.5. In some embodiments, an AICA riboside solution is co-formulated with one or more additional therapeutic agents.

In addition, kit 101 can optionally also include a second container 104. The second container 104 comprises a sterilized perfusate solution of AICA riboside (or analog thereof) and can be administered in vivo or ex vivo. The AICA riboside in the perfusate solution can be lyophilized or non-lyophilized. The perfusate solutions herein are preferably buffered for a controlled pH. In some embodiments, a perfusate solution herein has pH 6-8, or more preferably pH 6.5-7.5 or more preferably about 7.0. In some embodiments, the perfusate solutions herein are co-formulated with one or more additional therapeutic agents.

First container 102 and optionally the second container 104 are provided with a set of instructions for use 103. The instructions for use provide, for example, instructions for a physician to administer the compositions in first container 102 and/or second container 104 to a patient to treat or prevent a condition. Examples of conditions treatable or preventable by the compositions herein include an ischemic condition, a condition regulated by adenosine, or a condition associated with reduced blood flow to a tissue. In preferred embodiments, the condition being treated or prevented is a myocardial infarction, stroke, or death.

In some embodiments, instructions of use 103 provide methods for administering the compositions/solution herein, e.g., by intravenous (i.v.) administration or topically via a perfusate. Instructions for use 103 can also teach the effective amount of the compositions herein that is to be administered for. For example, instructions for use 103 can provide that AICA riboside is to be administered for a 7 hour period beginning 15 minutes before induction of anesthesia in a bypass operation. Instructions for use 103 can also include teach the proper dilution and/or dosing of AICA riboside. In some embodiments, when AICA riboside is obtained in a container comprising 1 g AICA riboside, and it needs be diluted to a final solution of depending on the concentration that it is administered in to a patient.

Instructions for use 103 can further provide instructions on how to administer such perfusate e.g., via a syringe or other suitable delivery device 105.

Instructions for use 103 can further provide that the compositions/solutions herein can be administered a patient undergoing surgery. The patient is preferably a high risk patient e.g., a female, having one or more prior myocardial infarctions, having one or more prior stokes, being over the agent of 60 or 70, having an unstable angina or recent myocardial infarction, or having an acute coronary stent failure.

The kits may be marketed and/or sold by a business for the treatment of any of the above or other conditions.

Compositions

The present invention relates to compositions comprising, consisting essentially of, or consisting of AICA riboside or an analog, solvate, isomer, tautomer, metabolite, prodrug, or salt thereof. AICA riboside, as used herein, refers to a compound of formula I.

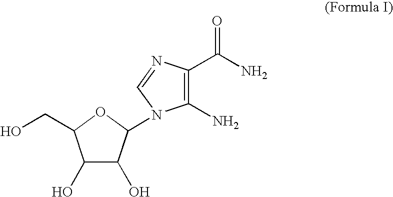

Examples of analogs of AICA riboside include those compounds having formula II: 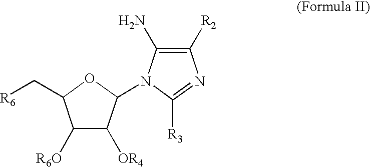
wherein R₂ is selected from the group consisting of hydrogen, —CN and the group 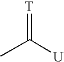
where T is selected from oxygen, sulfur, NOH, NH, and NO(CH₂)ₙCH₃ where n is from 0 to 2 and U is selected from lower alkoxy, amino, a 3 to 6 member heterocyclic ring optionally fused to a 3 to 6 member aryl ring, and the group 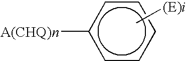
wherein A is one of NH and S, n is from 0 to 3, i is from 0 to 2, Q is one of hydrogen and hydroxy, and E represents a nitro or hydroxy group, provided that where U is amino, T is not one of sulfur, NOH, NH, and NOCH₃; where T is amino, U is not lower alkoxy; and where A is amino and n is 1, Q is not hydroxy;
R₃ is selected from hydrogen, halogen, and S—W, where W is phenyl, or substituted phenyl, or hydrogen when T is not oxygen and U is not amino;
R₄ and R₅ are each independently selected from hydrogen, —COCH₃ and lower alkyl, or together form a cyclic carbonate; and
R₆ is selected from, hydroxy, phosphate ester, —OSO₂NH₂, sulfhydryl, halogen, —OCOCH₃, —SCH₃, —SOCH₃, NH₂, and N₃;
provided that when R₂ is CONH₂, CONR-para-iodophenyl, hydrogen, CN, or CONHCH₂-φ and R₃ is hydrogen or halogen, and R₄ and R₅ are hydrogen, acyl, or together form a cyclic carbonate, then R₆ is not halogen, phosphate ester, OH, or —O-acyl wherein said compound
or any salt, solvate, isomer, tautomer, metabolite, analog, or prodrug thereof is at a concentration of between about 0.1 μM to about 500 μM, about 1 μM to about 400 μM, about 2 μM to about 300 μM, about 3 μM to about 200 μM, and about 4 μM to about 150 μM, preferably from about 5 μM to about 100 μM.

In a preferred embodiment, an AICA riboside analog has formula III: 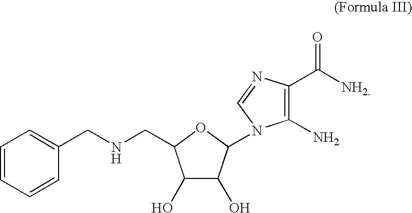
Pharmaceutical Formulations

The compositions described herein can be formulated in any manner known in the art, e.g., as a solution (i.v. or perfusate) or solid form. In some embodiments, the compositions herein are formulated as a solution, which allows for more rapid and quantitative absorption to the sites of action. In some embodiments, the compositions herein are formulated as a dry lyophilized material, which can be reconstituted as needed.

In any of the embodiments herein, compositions can be formulated for slow release and/or for administration via a drug eluting stent.

In any of the formulations herein, one or more composition may be present in an “effective amount”, i.e., in an amount effective to achieve therapeutic and/or prophylactic benefit.

Liquid formulations include, but are not limited to, solutions in which a composition of the invention is dissolved, emulsions comprising a composition of the invention, and a solution containing liposomes, micelles, or nanoparticles comprising a composition of the invention as disclosed herein. Preferably, the composition(s) herein are formulated into a solution for i.v. and/or perfusion.

Pharmaceutical formulations herein can optionally include one or more pharmaceutically acceptable carriers, excipients or diluents.

Formulations of the compositions herein will depend, in part, on the route of administration chosen. For example, the compositions herein can be delivered to a patient using routes including oral, buccal, topical, rectal, transdermal, transmucosal, subcutaneous, intravenous, inhalation (i.v.), and intramuscular applications, as well as ex vivo or in vivo application using a perfusate solution (e.g. cardioplegic perfusate solution).

In one embodiment, the compositions herein are buffered to maintain a set pH range (e.g., physiological pH). Buffers that can be used include, but are not limited to, sodium lactate, potassium chloride, Hanks' Balanced Salts, Earle's Balanced Salts, histidine buffer, Locke's solution and Tyrode's solution, and Ringer's solution, glycylglycine, citrate, Tris, phosphate buffer, phosphate-buffered saline (PBS), saline citrate, HEPES, NaCl, Bis-Tris, ADA, aces, PIPES, MOPSO, TES, DIPSO, MOBS, TAPSO, Trizma, HEPPSO, POPSO, TEA, EPPS, Tricine, Gly-Gly, Bicine, HEPBS, sodium phosphate, phosphate-bicarbonate buffer, and other phosphate buffers. In preferred embodiments, a composition herein is buffered to maintain a set pH range, wherein the buffer is selected from the group consisting of a Tris buffer, phosphate buffer, and a histidine buffer.

In some embodiments, the compositions, formulations, or preferably solutions herein are formulated within a pH range from about 6 to about 8, more preferably about 6.5 to about 7.5, or more preferably about 7.0. In other embodiments, the pH of a composition, formulation, or preferably solution herein is at least 5.0, 5.2, 5.4, 5.6, 5.8, 6.0, 6.2, 6.4, 6.6, 6.8, 7.0, 7.2, 7.4, 7.6, 7.8, or 8.0. In some embodiments, the pH of a composition herein or a formulation thereof has pH that is less than 9.0, 8.8, 8.6, 8.4, 8.2, 8.0, 7.8, 7.6, 7.4, 7.2, 7.0, 6.8, 6.6, 6.4, 6.2, or 6.0.

In some embodiments, compositions and/or formulations described herein are manufactured as lots, wherein a pH range of between 6 and 8, 6.5 and 7.5, or about 7 is present in more than 80%, more than 85%, more than 90%, more than 91%, more than 92%, more than 93%, more than 94%, more than 95%, more than 96%, more than 97%, more than 98%, more than 99%, or more than 99.99% of all the lots. Preferably, a pH range of between 6 and 8 in more than 80%, more than 85%, more than 90%, more than 91%, more than 92%, more than 93%, more than 94%, more than 95%, more than 96%, more than 97%, more than 98%, more than 99%, or more than 99.99% of all the lots.

The formulations herein may also include one or more excipients, for example, preservatives, solubilizers, fillers, lubricants, stabilizers, anti-microbial agents, albumin, and the like. Examples of the above are disclosed, for example, in Remington: The Science and Practice of pharmacy, 763-764 (Alfonso R. Gennaro ed., 20^{th} ed., Lippincott, Williams & Wilkins 2000).

A solution of the compositions(s) herein is preferably provided in a concentrated form suitable for dilution, in the range of about 10 mg/mL to about 80 mg/mL, about 20 mg/mL to about 70 mg/mL, about 30 mg/mL to about 60 mg/mL, or more preferably about 40 mg/mL. The 40 mg/mL solution can then be further diluted by a health care provider prior to administration to a patient. In preferred embodiments, the concentrated solution above is diluted to a total volume of 500 mL in normal saline prior to administration. Dilutions are performed by health care provider based on amount of drug to be administered over the course of treatment.

In some embodiments, the concentrated form further comprises sodium chloride in the range of about 0.1 mg/mL to about 10 mg/mL, about 0.5 mg/mL to about 8 mg/mL, about 1 mg/mL to about 7 mg/mL, about 2 mg/mL to about 6 mg/mL, about 3 mg/mL to about 5 mg/mL, preferably about 3.5 mg/mL to about 4 mg/mL, more preferably about 3.9 mg/mL.

In another preferred embodiment, the i.v. solution is diluted so as to provide a dosage to a patient in the range of greater than about 0.001 mg/kg/minute, greater than about 0.01 mg/kg/minute to less than about 10 mg/kg/minute, greater than about 0.1 mg/kg/minute to less than about 5 mg/kg/minute, greater than about 0.5 mg/kg/minute to less than about 3 mg/kg/minute, greater than about 1 mg/kg/minute to less than about 2 mg/kg/minute, preferably greater than about 0.05 mg/kg/minute to less than about 0.2 mg/kg/minute, and more preferably about 0.1 mg/kg/minute.

In one preferred embodiment, the i.v. solution is diluted so as to provide a total dosage of 1 mg/kg to 500 mg/kg, 2 mg/kg to 400 mg/kg, 3 mg/kg to 300 mg/kg, 4 mg/kg to 250 mg/kg, 5 mg/kg to 225 mg/kg, 10 mg/kg to 200 mg/kg, 30 mg/kg to 160 mg/kg. In some embodiments, the total dosage is about 40 mg/kg for a patient undergoing cardiac surgery as described herein or 120 mg/kg for a patient undergoing non-cardiac surgery as described herein.

In some embodiments, a composition, formulation or solution herein is provided so as to provide a blood plasma concentration of the composition in the patient of 0.01 μg/mL to 50 μg/mL, 0.1 μg/mL to 45 μg/mL, 1 μg/mL to 35 μg/mL, 2 μg/mL to 30 μg/mL, 3 μg/mL to 25 μg/mL, 4 μg/mL to 20 μg/mL, 5 μg/mL to 15 μg/mL, 6 μg/mL to 10 μg/mL, or 7 μg/mL to 9 μg/mL. In some embodiments, a composition, formulation, or solution herein is provided so as to provide a blood plasma concentration of 1 μg/mL to 20 μg/mL, 3 μg/mL to 6 μg/mL, or about 5 μg/mL.

In one embodiment, the present invention contemplates a perfusate solution comprising a composition described herein. The perfusate solution may have a concentration of a composition herein (preferably AICA riboside or analog thereof having formula III) in the range of about 0.1 μM to about 500 μM, about 0.5 μM to about 400 μM, about 1 μM to about 300 μM, about 2 μM to about 200 μM, about 3 μM to about 150 μM, about 4 μM to about 125 μM, preferably about 5 μM to about 100 μM, more preferably about 20 μM. In a preferred embodiment, a composition of the present invention or a formulation thereof is administered as a perfusate solution to a patient undergoing CABG surgery or an organ transplant such that the concentration of AICA riboside, including compounds of formula I, II, and III or analogs thereof is 20 μM or 5 μg/mL.

In a preferred embodiment, a perfusate solution of the invention comprises a concentrated form of the compositions herein in a range of about 0.01 mg/mL to about 30 mg/mL, about 0.1 mg/mL to about 20 mg/mL, about 0.5 mg/mL to about 10 mg/mL, or more preferably about 1 mg/mL.

In some embodiments a perfusate solution may have a concentration of a composition herein of 0.1 μM to 500 μM, 0.5 μM to 400 μM, 1 μM to 300 μM, 2 μM to 200 μM, 3 μM to 150 μM, 4 μM to 125 μM, or 5 μM to 100 μM, 6 μM to 90 μM, 7 to 80 μM, 8 μM to 70 μM, 7 μM to 60 μM, 8 μM to 50 μM, 9 μM to 40 M, or 10 μM to 30 μM. Preferably, a concentration of a composition herein in a perfusate solution is about 20 μM.

Solid pharmaceutical formulations include, but are not limited to, lyophilized powders, tablets, dispersible granules, capsules, sachets, and suppositories. Excipients may also be added to such solid formulations including, but not limited to, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The formulations may also be made as formulated as a sustained release preparation.

In some embodiments, formulations of the present invention may exert local and regional prophylactic or therapeutic effects when administered locally at or near particular sites of tissue damage related to a condition described herein. For example, formulations of the present invention may be used to treat and/or prevent an ischemic condition, a condition modulated by adenosine; a condition associated with reduced blood flow to a tissue; or simply prevents morbidity/mortality in patient.

In a preferred embodiment, the compositions herein are administered perioperatively. For example, an i.v. solution comprising, consisting essentially of, or consisting of a composition herein can be administered beginning between 1-90 minutes before anesthesia, 2-80 minutes before anesthesia, 3-70 minutes before anesthesia, 4-60 minutes before anesthesia, 5-50 minutes before anesthesia, 6-40 minutes before anesthesia, 7-30 minutes before anesthesia, 8-28 minutes before anesthesia, 9-26 minutes before anesthesia, 10-24 minutes before anesthesia, 11-22 minutes before anesthesia, 12-20 minutes before anesthesia, 13-18 minutes before anesthesia, 14-16 minutes before anesthesia, or preferably 15 minutes before anesthesia.

The formulations and/or compositions herein can be administered for a period of 1 to 24 hours, 2 to 20 hours, 3 to 16 hours, 4 to 12 hours, 5 to 10 hours, 6 to 8 hours, or preferably 7 hours.

In a preferred embodiment, the formulations and/or compositions herein are administered post-surgery for a period of between 1 to 36 hours, 2 to 32 hours, 3 to 28 hours, 4 to 24 hours, 5 to 20 hours, 6 to 18 hours, 7 to 16 hours, 8 to 14 hours, or 9 to 12 hours.

In preferred embodiments, the formulations and/or compositions herein are administered beginning 15 minutes prior to administration of anesthesia for cardiac surgery (e.g. CABG), and continuing for 7 hours. However, the rate and time of dosage may be altered depending on a number of variables, not limited to the activity of the composition used, the condition to be treated, the mode of administration, the requirements of the individual subject, the severity of the condition being treated, and the judgment of the practitioner.

In another embodiment, the formulations and/or compositions herein are administered to treat a gastrointestinal condition. In such embodiment, the compositions are formulated to be delivered orally- or rectally via solutions, suspensions, ointments, enemas and/or suppositories comprising one or more compositions of the present invention. For example to treat a condition related to the gastrointestinal system, a suppository formulation comprising a composition of the present invention can be used. In such an embodiment, a formulation described herein would produce a benefit locally at or near the site of application, rather than systemically, by preventing or reducing adverse effects associated with a condition described herein. In a preferred embodiment, a formulation comprising a composition disclosed herein is useful in treating acute bowel ischemia.

Combinations

The compositions herein may be co-administered or co-formulated with one or more additional therapeutic agents. The choice of therapeutic agents that can be co-formulated with or co-administered with the compositions of the invention will depend, in part, on the condition being treated or prevented.

In some embodiments, a composition herein is co-formulated or co-administered with an adenosine deaminase inhibitor, a blood-clotting inhibitor, an anticoagulant, an anti-platelet agent, an anti-hypertensive agent, a cholesterol lowering drug, a vasodilator, a beta-blocker, an ace-inhibitor, an analgesic, an anti-inflammatory agent, an anti-neoplastic agent, and/or a diuretic.

In a preferred embodiment, a composition herein is co-formulated or co-administered with an adenosine deaminase inhibitor. Such an inhibitor can prevent an adenosine deaminase from catalyzing the deamination of adenosine to inosine. (See Law, U.S. Pat. No. 6,103,702).

Examples of adenosine deaminase inhibitors that may be combined with a compositions herein include but are not limited to 9-(1-hydroxy-2-octyl)adenine, erythro-9-(2-hydroxy-3-nonyl)adenine (EHNA), 2′-deoxycoformycin, coformycin, 1,3,7-trimethylxanthine (caffeine), and pentostatin. In a preferred embodiment, the present invention contemplates the co-administration or co-formulation of a composition as described herein with pentostatin. More preferably, the present invention relates to a composition or formulation thereof comprising a composition comprising AICA riboside, including compounds of formula I, II, and III or an analog thereof and pentostatin.

In one aspect of the present invention, the compositions described herein may be co-administered or co-formulated with a blood clotting inhibitor. The blood clotting inhibitor of the present invention can be any drug, agent or pharmaceutical composition that prevents or inhibits blood clotting. The inhibitor can act by preventing or inhibiting blood clot formation by any of a variety of mechanisms including reduction of blood clotting factors or reducing platelet activation or aggregation, or mitigating the effects of instigating factors, such as inflammation or stress. The blood clotting inhibitor can also act by breaking down or dissolving a blood clot after formation. There are several classes of blood clotting inhibitor, including antiplatelet agents, thrombolytic enzymes, aggregation inhibitors, glycoprotein IIb/IIIa inhibitors, glycosaminoglycans, thrombin inhibitors, anticoagulants, heparins, low molecular weight heparins, coumarins, indandione derivatives and tissue plasminogen activators. See, The Physicians' Desk Reference (56^{th} ed., 2002) Medical Economics; Mosby 's Drug Consult, 2002, Elsevier Science; Goodman and Gilman's The Pharmacologic Basis of Therapeutics, (9^{th} ed. 1996) Pergamon Press; Drug Facts and Comparisons, updated monthly, September, 2002, Facts and Comparisons, Wolters Kluwer Company, St. Louis, Mo.

For the purposes of this invention, any substance that prevents or inhibits the formation of blood clots or dissolves or breaks down a blood clot is suitable. Such a blood clotting inhibitor can be, for example, cilostazol (PLETAL®, Otsuka), clopidogrel (PLAVIX®, Sanofi-Aventis), ticlopidine (TICLID®, Syntex), tirofiban (AGGRASTAT®, Merck), eptifibatide (INTEGRILIN®, COR Therapeutics), abciximab (REOPRO®, Eli Lill y), anagrelide (AGRYLIN®, Roberts), dipyridamole (PERSANTIN®, Boehringer Ingelheim), aspirin (ECOTR®, and others), dipyridamole/aspirin (AGGRENOX®, Boehringer Ingelheim), dalteparin (FRAGMIN®, Pharmacia), enoxaparin (LOVENOX®, Aventis), tinzaparin (INNOHE®, DuPont), heparin (various), danaparoid (ORGANON®, Organon), antithrombin III (THROMBATE®, Bayer), lepirudin (REFLUDAN®, Sanofi-Aventis), argatroban (ACOVA®, Glaxo SmithKline), bivalirudin (ANGIOMAX®, Medicines Company), warfarin (COUMADIN®, DuPont) anisidione (MIRADON®, Schering), alteplase (ACTIVASE®, Genetech), reteplase (RETAVASE®, Boehringer Mannheim), tenecteplase (TNKASE®, Genentech), drotrecogin (XIGRIS®, Eli Lilly), anistreplase (EMINASE®, Roberts), streptokinase (STREPTASE®, Astra), urokinase (ABBOKINASE®, Abbott) and combinations thereof.

In some embodiments, the composition(s) herein are co-formulated or co-administered with a blood clotting inhibitor. Preferably, such blood clotting inhibitor is aspirin.

In some embodiments, the composition(s) herein are co-formulated or co-administered with an anti-neoplastic agent. Examples of anti-neoplastic agents include, but are not limited to, Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin ; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride ; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Imofosine; Interferon Alfa-2a; Interferon Alfa-2b ; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingbl; Safingol Hydrochloride; Semustine; Simtrazene; Sparfosate Sodium; Sparsomycinl, Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazoftirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Sterilization

Any of the compositions and formulations herein are preferably sterilized by any means known in the art.

In some embodiments, sterilization or sterilizing involves subjecting any composition or formulation herein to a set of sterilization exposure conditions over a period of time such that a surviving microbial population is reduced. In some other embodiments, sterilization or sterilizing involves reducing in a composition of the present invention a surviving microbial population by a factor of greater than 50%, by a factor of greater than 60%, by a factor of greater than 70%, by a factor of greater than 80%, by a factor of greater than 90%, by a factor of greater than 99%, or by a factor of greater than 99.99%.

In some embodiments, the compositions herein are sterilized to reduce the presence of an unwanted material by a factor of greater than 50%, by a factor of greater than 60%, by a factor of greater than 70%, by a factor of greater than 80%, by a factor of greater than 90%, by a factor of greater than 99%, or by a factor of greater than 99.99%.

In another embodiment, the invention provides sterilized compositions as described herein having an impurity of between about less than about 1% by weight to about less than 10% by weight, about less than 2% by weight to about less than 9% by weight, about less than 3% by weight to about less than 8% by weight, about less than 4% by weight to about less than 7% by weight, about less than 5% by weight to about less than 6% by weight, preferably about less than 1%, more preferably about less than 0.9%, 0.8%, 0.7%, 0.6%, 0.5%, 0.4%, 0.3%, 0.2%, or 0.1%. Any of the sterilized compositions contemplated herein have preferably less than 50%, 40%, 30%, 20%, 10%, 9%, 8%, 7%, 6%, 5%, 4%, 3%, 2%, 1%, 0.5%, or 0.1% impurity by weight.

Sterilization can be achieved by various methods known in the art. Compositions and formulations of the present invention may be sterilized by several sterilization techniques including lyophilization, steam sterilization, dry heat sterilization, chemical “cold” sterilization, filtration sterilization, radiation sterilization, or a combination thereof

In one embodiment, a composition or formulation herein is sterilized by lyophilization methods or by non-lyophilization methods. For example, the present invention contemplates sterilized, lyophilized AICA riboside and analogs thereof.

Lyophilization may be conducted by any method known in the art. For example, a general procedure for lyophilization may include a first step of dissolving any of the compositions of the present invention in a suitable solvent. The second step may involve sterilizing the bulk solution via a bacteria rententive filtration. The third step may include filling individual sterile containers and partially stoppering the containers under aseptic conditions. The fourth step can comprise transporting the stoppered containers to a lyophilizer and loading them into the chamber under aseptic conditions. The fifth step can comprise freezing the solution by placing the containers on cooled shelves in a freeze-drying chamber or pre-freezing another chamber. The sixth step may include applying a vacuum to the chamber under aseptic conditions. The seventh step can comprise completely stoppering the vials by a hydraulic or screw top stoppering mechanism installed in the lyophilizer.

Preferred embodiments of lyophilization include (i) loading a chamber with the composition or formulation to be sterilized (e.g. AICA riboside or analog thereof); (ii) ramping temperature from 25° C. to ≦−40° C.; leaving the product at =31 40° C. for 10 hours to freeze; (iii) evacuating the chamber; (iv) drying the composition for 24-28 hours at a temperature that is ramped from −40° C. to 45° C.; (v) maintaining a vacuum in the chamber (preferably at 100-200 microns); (vi) performing a second drying step at 45° C. for 38 hours; (vii) equilibrating the chamber to 25° during 4 hours prior to stoppering; (iix) bleeding the chamber with a filtered nitrogen; (ix) maintaining vacuum at <500 microns; (x) stoppering vials automatically in the sealed lyophilizer; and (xi) sealing vials with an aluminum overseal.

In one embodiment, the compositions and formulations herein are sterilized and non-lyophilized.

Examples of methods for providing sterilized, non-lyophilized compositions and formulations include, but are not limited to, steam sterilization, dry heat sterilization, chemical cold sterilization, filtration sterilization, radiation sterilization, or a combination thereof.

In one embodiment, a composition or formulation herein is sterilized by steam sterilization. Steam sterilization may involve, for example, the inmmersion of a composition in pressurized steam at a temperature, which has the effect of degrading microorganisms at a rate proportional to the amount of time the preparation is treated. A composition may be sterilized by super-heated steam or by moist saturated steam sterilization at a temperature of about 100° C. to about 200° C., about 105° C. to about 175° C., about 110° C. to about 150° C., about 115° C. to about 140° C., about 120° C. to about 130° C., preferably about 121° C. Such temperatures can be achieved using an autoclave. The types of autoclaves that may be used are saturated steam autoclaves, superheated water spray autoclaves, and air over steam autoclaves. See Barry D. Garfinkle & Martin Henley, Sterilization, in Remington: The Science and Practice ofPharmacy, 755 (Alfonso R. Gennaro ed., 20^{th} ed., Lippincott, Williams & Wilkins 2000). Thus, a composition herein may be steam sterilized at a sufficient temperature and for a sufficient time to reduce any microbial contamination or unwanted material to a suitable level and still provide an active and stable composition.

In another embodiment, any of the compositions or formulations herein may be sterilized by dry heat sterilization. This form of sterilization may involve the destruction of microorganisms, as well as any chemical activity of their by-products, including endotoxins or pyrogens, and any other unwanted material present in a preparation of the present invention. Sterilization by dry heat may be conducted at a temperature from about 100° C. to about 200° C., about 120° C. to about 190° C., about 140° C. to about 180° C., and preferably from about 120° C. to about 160° C. and at a time from about 10 minutes to about 240 minutes, about 20 minutes to about 200 minutes, and preferably from about 30 minutes to about 180 minutes. More preferably, dry heat sterilization is conducted at a termperature from about 250° C. for about 30 to about 60 minutes. This technique may be performed using different autoclaves, including a dry heat batch sterilizer and a dry heat tunnel apparatus. See Barry D. Garfinkle & Martin Henley, Sterilization, in Remington: The Science and Practice of Pharmacy, 763-764 (Alfonso R. Gennaro ed., 20^{th} ed., Lippincott, Williams & Wilkins 2000). Thus, a composition or formulation of the present invention may be dry heat sterilized at a sufficient temperature and for a sufficient time to reduce any microbial contamination or any other unwanted material to a suitable level and still provide an active and stable composition.

In another embodiment chemical cold sterilization is used to sterilize a composition or formulation of the invention. Chemical cold sterilization may involve the treatment of compositions with agents that eliminate microorganisms or any other unwanted material present in a composition. Agents that may be used include gases or vapors of chlorine dioxide, ethylene oxide, propylene oxide, formaldehyde, betapropiolactone, ozone, hydrogen peroxide, peracetic acid, or a combination thereof. See Barry D. Garfinkle & Martin Henley, Sterilization, in Remington: The Science and Practice of Pharmacy, 765-770 (Alfonso R. Gennaro ed., 20^{th} ed., Lippincott, Williams & Wilkins 2000). In one embodiment, a composition contemplated herein is sterilized by chemical cold sterilization using ethylene oxide (EtO) at a concentration range of between about 50 mg/L to about 2000 mg/L, about 100 mg/L to about 1800 mg/L, about 200 mg/L to about 1600 mg/L, about 300 mg/L to about 1400 mg/L, or about 400 mg/L to about 1200 mg/L using a subatmospheric sterilizer. In another embodiment of the present invention, a composition contemplated herein is sterilized by hydrogen peroxide gas. In a preferred embodiment, the invention contemplates any of the compositions described herein sterilized by chlorine dioxide (CD). Thus, a composition of the invention may be chemical cold sterilized using an appropriate agent for a sufficient time to reduce any microbial contamination to a suitable level and still provide an active and stable composition.

In another embodiment, filtration sterilization may be used to sterilize a composition or formulation of the present invention. Filter sterilization involves the removal of microorganisms, organic matter, particulate matter and any other unwanted material from a liquid preparation as it passes through a filter. Filtration sterilization is based on the concept of removing microorganisms and any other unwanted material that may be present in the preparation. Particulate matter may be removed by filtration through one or more of the following: sieving or screening, entrapment or impaction, and electrostatic attraction. In one embodiment a composition or formulation herein may be sterilized using a filter having a pore size from about 0.01 to about 5 μm, about 0.02 μm to about 4.5 μm, about 0.03 μm to about 4 μm, about 0.04 μm to about 3.5 μm, about 0.05 μm to about 3 μm, about 0.1 μm to about 2.5 μm, about 0.2 to about 2 μm, about 0.3 μm to about 1.5 μm, about 0.4 μm to about 1 μm, and about 0.5 μm to about 0.9 μm, or from about 0.2 μm to about 0.5 μm. In a preferred embodiment, compositions or methods are filtered using a filter from about 0.22 μm to about 0.45 μm.

The filters may be manufactured from a variety of polymers, including nylon, polysulfone, polycarbonate, polyvinylidiene difluoride, polyvinylidiene fluoride (PVF), cellulosic esters (MCE), and polytetrafluouroehylene (PTFE), and the like. See Barry D. Garfinkle & Martin Henley, Sterilization, in Remington: The Science and Practice of Pharmacy, 770-771 (Alfonso R. Gennaro ed., 20^{th} ed., Lippincott, Williams & Wilkins 2000). In some embodiments, filtration sterilization is used to sterilize hyperalimentation solutions, extemporaneously compounded preparations, and more preferably intravenous (i.v.) admixtures. Thus, a composition of the invention may be filtration sterilized using an appropriate membrane having an appropriate pore size to reduce the level of any microbial contaminants or of any other unwanted material to a suitable level and still provide an active and stable composition.

In another embodiment radiation sterilization may be used to sterilize a composition or formulation of the invention. Radiation sterilization involves the bombardment of an object with radiation, including electromagnetic radiation or particle radiation. In one preferred embodiment the composition is sterilized by electromagnetic radiation, including ultraviolet, X-ray, cosmic radiation, and most preferably gamma radiation. In a preferred embodiment a composition is sterilized by cobalt-60, more preferably where the amount of cobalt-60 used is from about 1 MCi to about 20 MCi, about 2 MCi to about 18 MCi, about 3 MCi to about 16 MCi, and about 3.5 MCi to about 14 MCi, preferably about 4 MCi to about 12 MCi.

Particulate radiation, also known as corpuscular radiation, is radiation by particle bombardment. In one preferred embodiment, the invention contemplates a composition or formulation herein sterilized by particle radiation, including proton particles, neutron particles, preferably electron particles. The dosage of radiation selected for sterilization by particle radiation may be from about 1 kGy (kilogray, 1 Gy=1 joule/kg) to about 25 kGy, 5 kGy to about 20 kGy, about 10 kGy to about 15 kGy, preferably from about 15 kGy to about 25 kGy, and more preferably from about 2 kGy to about 8 kGy. In a preferred embodiment, the particle radiation by electron bombardment may be applied to a composition or formulation herein using an electron accelerator, including an alternating current machine and a direct-current machine.

Radiation sterilization may therefore be useful to provide compositions of the invention. For example, a composition of the invention may be radiation sterilized using an appropriate radiation type and dosage to reduce any microbial contamination to a suitable level and still provide an active and stable composition. Methods

In one embodiment, the compositions and formulations herein can be used to modulate or increase the level of local endogenous adenosine. Adenosine (Ad) may bind one or more of its known receptors (Adr) including A₁, A_{2A}, A_{2B}, and A₃, which may modulate various physiological responses that affect conditions, such as e.g. ischemic conditions. For example, adenylate cyclase may be activated where a stimulatory G protein binds an adenosine receptor, thereby leading to cAMP production and the activation of kinases ultimately resulting in vasodilation. Adenosine binding may also inhibit the physiological response where an inhibitory G protein binds an adenosine receptor, thereby leading to an inhibition of adenylate cyclaseb.

In one embodiment, the compositions and formulations herein can be used to treat and/or prevent a condition in a patient. Conditions that can be treated/prevented by the compositions and formulations herein include, for example, ischemic conditions, condition regulated by adenosine or an adenosine receptor, a conditions associated with reduced blood flow to a tissue, undesirable or uncontrolled cell proliferation, or death.

Examples of ischemic conditions include, ischemic heart diseases such as myocardial infarction, angina pectoralis, atherosclerotic injury, stroke, cerebral vasoconstriction, and cramps; ischemic muscle injury associated with muscle spasm; ischemic brain condition, and ischemic GI/bowel condition.

Examples of a condition of undesirable or uncontrolled cell proliferation include, but are not limited, tumors, various types of cancers such as primary tumors and tumor metastasis, restenosis (e.g. coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

In particular, the present invention relates to the use of the compositions, formulations and stents herein to treat cancer. Examples of cancer include leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic comeal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

In some embodiments, a condition treated/prevented by the compositions herein is vascular, while in other embodiments it is non-vascular.

Examples of vascular conditions that may be treated or prevented by the compositions or formulations herein include, but are not limited to those vascular conditions caused by myocardial ischemia, a heart attack, a stroke, a transmural or non-transmural myocardial infarction, an acute myocardial infarction, coronary artery disease, coronary heart disease, an arrhythmia, sudden cardiac death, a cerebrovascular accident such as stroke, congestive heart failure, a life-threatening dysrhythmia, cardiomyopathy, a transient ischemic attack, an acute ischemic syndrome, or angina pectoralis, acute coronary stent failure, or a combination thereof. In a preferred embodiment, the compositions herein are used to treat and/or prevent a myocardial infarction or a stroke in a patient.

In some embodiments, vascular conditions that may be treated or prevented by the compositions or formulations herein include conditions caused by gastro-intestinal or mesenteric ischemia/infarction, microvascular disease of diabetes mellitus (which can affect the brain, the kidney, the heart, the skin, the retina, and the peripheral nerves and their associated microvasculatures), and events resulting in a less prolonged loss of blood flow, such as chronic bowel ischemia, acute bowel ischemia, kidney ischemia, intermittent claudication of skeletal muscle, migraine headaches, Raynaud's phenomenon, or a combination thereof.

In another embodiment, a condition that may be treated or prevented by the compositions or formulations herein may be a non-vascular condition. Examples of a non-vascular condition that may be treated or prevented by the methods herein include but are not limited to hepatic injury, pancreatic injury, disseminated intravascular coagulation such as due to bowel ischemia, shock, and death from non-cardiac causes. In a preferred embodiment, the compositions herein are used to treat and/or prevent shock in a patient.

The compositions of the present invention may be administered to a patient to treat and/or prevent an adenosine receptor-related condition. An adenosine receptor-related condition is one where the activity of an adenosine receptor is implicated. Specifically, the condition may be treated by promoting the binding of adenosine to its receptor thereby increasing the activity of adenylate cyclase or a kinase activated due to the increased activity of adenylate cyclase. The increased activity of adenylate cyclase or one of its substrates may lead to increased vasodilation. An adenosine receptor-related condition may also be treated and/or prevented by inhibiting the binding of adenosine to its receptor. Preferably, the present invention provides a method for treating and/or preventing a condition in a patient comprising administering a composition or formulation thereof described herein wherein the condition is an adenosine receptor-related condition. An adenosine receptor-related condition is one in which the activity of an adenosine receptor is implicated, either through inhibition or through activation of the receptor.

In one other embodiment, the condition may be an adenosine receptor-related condition. Such conditions include, but are not limited to, asthma, allergies, allergic diseases (e.g. allergic rhinitis and-sinusitis), autoimmune diseases (e.g. lupus), diarrheal diseases, insulin resistance, diabetes, prevention of mast cell degranulation associated with ischemia/reperfusion injuries, heart attack, inflammatory condition, thrombotic condition (e.g., pulmonary embolism, acute thrombosis of the coronary arteries, myocardial infarction, acute thrombosis of the cerebral arteries (stroke) or other organs), inhibition of angiogenesis in neoplastic tissues, and inhibition of angiogenesis in diabetic retinopathy or hyperbaric oxygen-induced retinopathy.

The methods of treating and/or preventing a condition described herein involve administering a composition or formulation of the present invention to a patient in an effective amount.

An effective amount may be such that it maintains the blood plasma concentration of a composition of the invention from greater than about 0.01 μg/mL to less than about 50 μg/mL, greater than about 0.1 μg/mL to less than about 45 μg/mL, greater than about 1 μg/mL to less than about 35 μg/mL, greater than about 2 μg/mL to less than about 30 μg/mL, greater than about 3 μg/mL to less than about 25 μg/mL, greater than about 4 jig/mL to less than about 20 μg/mL, greater than about 5 μg/mL to less than about 15 μg/mL, greater than about 6 μg/mL to less than about 10 μg/mL, greater than about 7 μg/mL to less than about 9 μg/mL, preferably greater than about 1 μg/mL to less than about 20 μg/mL, more preferably greater than about 3 μg/mL to less than about 6 μg/mL, and most preferably about 5 μg/mL. Under one embodiment, the desired blood plasma concentration of AICA riboside in a patient is achieved after administration within about 1 minute to about 15 minutes, about 2 minutes to about 12 minutes, about 3 minutes to about 10 minutes, about 4 minutes to about 8 minutes, about 5 minutes to about 7, preferably about 2 minutes to about 5 minutes.

In another embodiment, an effective amount of a composition herein is 0.001 mg/kg/minute to 20 mg/kg/minute, 0.005 mg/kg/minute to 10 mg/kg/minute, 0.01 mg/kg/minute to 5 mg/kg/minute, 0.05 mg/kg/minute to 1 mg/kg/minute, 0.1 mg/kg/minute to 0.5 mg/kg/minute, and more preferably about 0.1 mg/kg/minute.

In one aspect, the present invention provides a method of treating and/or preventing a condition described herein by administering a composition or formulation of the present invention at a dose of 1 mg/kg to 500 mg/kg, 2 mg/kg to 400 mg/kg, 3 mg/kg to 300 mg/kg, 4 mg/kg to 250 mg/kg, 5 mg/kg to 225 mg/kg, 10 mg/kg to 200 mg/kg, 30 mg/kg to 160 mg/kg. In some embodiments, the total dosage is about 40 mg/kg. When administered to a patient undergoing a non-cardiac surgery the total dose may be at about 100-240 mg/kg.

In any of the embodiments herein, the compositions or formulations may be administered for a period of greater than about 1 minute to less than about a year. In preferred embodiments, such compositions or formulations thereof are administered for a period of time greater than about 1 hour to less than about 1 week, a period of time greater than about 2 hours to less than about 1 day, a period of time greater than about 3 hours to a less than about 18 hours, a period of time greater than about 4 hours to less than about 12 hours, a period of time greater than about 6 hours to less than about 10 hours, and more preferably for a period of time greater than about 4 hours to less than about 8 hours.

Lower or higher doses than those disclosed herein may be used, as required. Such dosages, however, may be altered depending on a number of variables, not limited to the activity of the compound used, the condition to be treated, the mode of administration, the requirements of the individual subject, the severity of the condition being treated, and the judgment of the practitioner. The foregoing ranges are merely suggestive, as the number of variables in regard to an individual treatment regime is large, and considerable excursions from these recommended values are not uncommon.

In another embodiment, the present invention provides a method for treating and/or preventing a condition described herein in a patient undergoing surgery. The surgery can be cardiac or non-cardiac.

Examples of cardiac surgeries include, but are not limited to, bypasses, such as coronary artery bypass grafting (CABG), percutaneous transluminal coronary angioplasty (PTCA), percutaneous transluminal angioplasty (PTA), laser angioplasty, cerebral angioplasty, an atherectomy, an intravascular stent procedure, carotid endarterectomy, heart and heart-lung transplant, implantation of artificial heart devices and defibrillators, valve replacement or repair, and congenital surgery, and the like.

In a preferred embodiment, a composition or formulation herein is administered to a patient undergoing a CABG surgery. Preferably, a buffered solution of AICA riboside at pH 6-8, or 6.5 and 7.5 is administered to a patient undergoing CABG surgery at a rate of 0.1 mg/kg/minute for about 7 hours by i.v. injection and optionally using a perfusate solution of about 5 μg/mL or about 20 μM.

In one embodiment, a cardiac surgery is an intravascular stent procedure. An intravascular stent is a device adapted to be implanted into the blood vessel or coronary artery of a patient. Generally, stents are cylindrical devices capable of holding open and sometimes expanding a segment of a blood vessel or a coronary artery. Stents may be delivered in a compressed form to a target location and then deployed in an expanded form to support the vessel or artery and prevent a reclosure. Stents may be used to treat atherosclerotic stenosis in an artery and/or blood vessel or to treat and repair blood vessels following a narrowing or stenosis in the artery or blood vessel.

Preferably under the present invention, the cardiac surgery may involve the use of a stent to treat and/or repair blood vessels after a stenosis has been compressed by a PTCA or a PTA, or after a stenosis has been removed by an atherectomy by improving the result of the procedure and/or reducing the possibility of a reclosure or restenosis. The cardiac surgery may also involve the use of a stent to compress a stenosis without an initial procedure such as a PTCA or a-PTA. Under the present invention, the surgery may involve the implantation of a stent on another body lumen such as carotid arteries, peripheral vessels, urethra, esophagus and bile duct. The cardiac surgery related to a vascular repair device may be an angioplasty procedure.

In one embodiment, the compositions of the present invention may be used in a drug-coated or drug-eluting stent where the outer portion of a standard intravascular stent is coated with a drug. Preferably, a polymer containing a composition described herein or a formulation thereof may be used to coat an intravascular stent. Following deployment of the stent, the compositions herein may diffuse out into the wall of the vessel or artery over the time following cardiac surgery to reduce and/or prevent a condition, which can be treated and/or prevented by increasing the endogenous localized level of extracellular adenosine.

In another embodiment, the surgery is non-cardiac or ambulatory.

Examples of non-cardiac or ambulatory surgery include, but are not limited to, small and large bowel resection, organ transplantation, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, organ transplant, thoracotomy, rhinoplasty, liposuction and the like.

The compositions or formulations herein can be administered perioperatively to a patient undergoing a surgery as described herein. In some embodiments, the composition may be administered before, during and/or after surgery. In some embodiments, the compositions are administered in a drug-eluting stent, an intravenous injection, a perfusate solution or a combination thereof. In some embodiments, the patient is also administered a second therapeutic agent. In a preferred embodiment, the second therapeutic agent is an adenosine deaminase inhibitor or a blood clotting inhibitor. In a more preferred embodiment, the second therapeutic agent is pentostatin or aspirin.

Screening

The present invention also contemplates screening an individual to determine the whether an individual is particularly susceptible to a condition as described herein that may be treated and/or prevented by the compositions or formulations described herein. Screening for such high risk patients may be performed using conventional clinical standards, such as a prior or contemporaneous diagnosis, family history of disease, or genetic screening. Thus, a person diagnosed as particularly susceptible to a condition, preferably a condition that is know to be treatable and/or preventable by modulating the level of endogenous localized extracellular adenosine may be administered a composition of the present invention or a formulation thereof to treat and/or prevent a condition described herein.

In one embodiment, a patient undergoing cardiac surgery is screened to determine whether the patient is a high risk patient. A physician may take a medical history to identify any high risk factors in a patient. One such high risk factor is a predisposition to complications from CABG surgery. Examples of high risk factors include, but are not limited to, elevated age such as 70 or older, unstable angina, a failed percutaneous transluminal coronary angioplasty, a decreased left ventricular function measured by an ejection fraction of less than about 40%, a decreased left ventricular function measured by an ejection fraction of less than about 30%, chronic or acute renal failure, dysrhythmia, a history of one or more prior myocardial infarctions, a history of one or more prior strokes, a history of one or more heart attacks, a prior myocardial infarction that occurred within about the last 24 months to about the last 48 months, or a combination thereof.

In a preferred embodiment, the patient is undergoing CABG surgery and is screened for a decreased left ventricular function measured by an ejection fraction of less than about 30% or less than about 40%. The ejection fraction may be measured by the method described in Example 1. Gruber et al., U.S. Pat. No. 5,817,640

In another embodiment, a patient undergoing non-cardiac surgery is screened for high risk factors. Examples of such factors include, but are not limited to, elevated age such as 65 to 70, atherosclerotic heart disease, i.e. coronary heart disease (evidenced by peripheral vascular disease or carotid artery diseases), diabetes, renal failure, heart failure currently under therapy, left ventricular hypertrophy & hypertension, hypertension for over 5 years, a myocardial infarction within 6 to 12 months prior to surgery, angina, arrhythmia, hypercholesterolemia, or a combination thereof. In a preferred embodiment, the patient being screened for high risk factors is undergoing organ transplantation surgery.

Kits

The present invention also relates to kits comprising one or more of the compositions herein in container(s) with written instructions for use thereof. FIG. 1 illustrates one example of a kit 101 of the present invention.

Kit 101 includes one or more first container(s) 102 comprising, consisting of, or consisting essentially of a composition or formulation herein for intravenous applications. In some embodiments, a first container 102 comprises a buffered solution of AICA riboside or an analog thereof (such as formula III). Such buffered solution is preferably at pH of 6-8 or more preferably 6.5-7.5, or more preferably about 7.0. Such buffered solution is preferably suitable for preparing an i.v. solution. Compositions or formulations in first container(s) 102 are preferably sterilized by lyophilization or non-lyophilization means. A composition in first container 102 can be in a concentrated solution form of 1 mg/mL to 100 mg/mL, 5 mg/mL to 90 mg/mL, 10 mg/mL to 80 mg/mL, 20 mg/mL to 70 mg/mL, 30 mg/mL to 60 mg/mL, or more preferably about 40 mg/mL. Such concentrated form can subsequently be used to pre a final i.v. solution or perfusate solution.

The kits 101 herein may optionally comprise one or more second container(s) 104 comprising a composition or solution of the present invention for preparing a perfusate solution. In one embodiment, second container 104 comprises a composition of the invention in a solution of water, saline solution, and/or perfusate solution. Preferably, the concentration of the solution of in a second container 104 is 0.01 mg/mL to 30 mg/mL, 0.1 mg/mL to 20 mg/mL, 0.5 mg/mL to 10 mg/mL, or more preferably about 1 mg/mL of one or more of the compositions herein.

In one embodiment, the first container(s) 102 and/or second container(s) 104 are ampules. In preferred embodiment, the container(s) are ampules suitable for holding a volume of about 0.01 mL to about 100 mL, about 0.1 mL to about 90 mL, about 0.5 mL to about 80 mL, about 1 mL to about 70 mL, about 2 mL to about 60 mL, about 3 mL to about 50 mL, about 4 mL to about 40 mL, about 5 mL to about 30 mL, about 6 mL to about 20 mL, and about 7 mL to about 10 mL. In more preferred embodiments, the first container(s) 102 is about a 20 mL ampule and the second container(s) 104 is about a 5 mL ampule.

Kit 101 can also include a set of instructions for use 103. A physician or other healthcare provider may follow the instructions for use 103 to prepare an i.v. solution or-perfusate solution from the solution of first container 102, suitable for administration to a patient. Instructions for use 103 can provide, for example, suitable diluents, such as water or saline solution that should be used to reconstitute or dilute compositions/formulation in container(s) 102. Instructions for use 103 can further provide appropriate final concentrations/dosages to be administered to a patient based on, in part, the condition being treated/prevented, age of patient, weight of patient, current condition of patient, type of surgery (if any) patient is undergoing, etc.

For example, a healthcare provider may follow instructions 103 to prepare an i.v. solution from the composition/solution in the first container 102 and to prepare a perfusate solution from the composition/formulation in the second container 104. In a preferred embodiment, instructions for use 103 can direct a healthcare provider to prepare a perfusate solution such that the final concentration of a composition herein is from 1, μM to 200 μM, 5 μM to 100 μM, 10 μM to 50 μM, or preferably about 20 μM. For example, the instructions 103 may provide that if 5 mL of 1 mg/mL AICA riboside is added to 995 mL perfusate solution, the resulting concentrate will be approximately 5 μg/mL or 20 μM. Moreover, instructions foruse 103 can direct a healthcare provider to prepare an i.v. solution having a final concentration of about 1 mg/mL to 100 mg/mL, 5 mg/mL to 90 mg/mL, 10 mg/mL to 80 mg/mL, 20 mg/mL to 70 mg/mL, 30 mg/mL to 60 mg/mL, or more preferably about 40 mg/ML.

The kit 101 may optionally comprise a third container (not shown) comprising, consisting of, or consisting essentially of a second therapeutic agent. In one embodiment, the composition or formulation is sterilized. The sterilized compositions or formulations may be lyophilized or non-lyophilized as described herein. In another embodiment, the third container comprises a composition comprising a second therapeutic agent having a set pH range as described herein. In a preferred embodiment, the invention contemplates a kit comprising a third container comprising a composition comprising an adenosine deaminase inhibitor. In a more preferred embodiments, the third container comprises pentostatin.

In use, a physician will formulate an i.v. solution based on the instructions 103 in the kit for solution 102. This will then be administered to a patient. During surgery the physician will apply to the heart or other relevant organ.

Business Methods

The present invention provides business methods for manufacturing, marketing and/or selling composition, formulation and kits as described herein.

In some embodiments, the compositions/formulations herein are manufactures in batches of at least 100 L, 200 L, 300 L, 400 L, 500 L, 600 L, 700 L, 800 L, or 900 L. Preferably, batch sizes will be between 10-900 L, 20-800 L, 30-700 L, 40-600 L, 50-500 L, or more preferably between 90 and 400L. Compounding of the compositions herein can include mixing a solution (e.g., water or saline) with the composition being manufactured and waiting until it dissolves. The solution can be heated to 30-35° C. to enhance dissolution. The solution can then be cooled to 20-25° C. and additional solution can be added with further mixing. The final concentration solution can then be sterilized, e.g., by running it through a pre-sterilized membrane, such as a 0.22 micron filter. Bulk solution is then filtered into clean depyrogenated vials or containers. A container of the present invention can have various volumes, but is preferably 50 mL in size. The vials and concentrated solutions therein are then sterilized by any one of the various means disclosed, e.g. lyophilization or non-lyophilization. In preferred embodiments, the business herein manufactures compositions/solutions that are lyophilized. Sterilized containers are then packaged, optionally into kits, and labeled for use and disclosed herein.

In one aspect, the present invention contemplates business methods that identify analogs of AICA riboside that may be used in the treatment/prevention of the conditions herein. The analogs may be identified internally within the business itself by research and development or the business may license or otherwise acquire the rights to analogs from another organization. The business methods herein can also optionally include the research and development of suitable formulations of AICA riboside or analogs thereof. Such formulations and reformulations can be accomplished by the business itself or by a third party licensed by the business.

In some embodiments, the business methods herein contemplate marketing/commercializing the one or more compositions, formulations or kits disclosed herein. To achieve this, the business may train a drug sales force to sell such products to potential users or healthcare providers (e.g., physicians, nurses, pharmacists, formulary officials). The sales force may add sales personnel or redirect existing sales personnel to sell the compositions, formulations, and/or kits herein to the appropriate buyers. The business herein may either market kits independently or in collaboration with a partner, such as a pharmaceutical company or biotechnology company. In some embodiments, the business herein may sell its rights to market the above compositions, formulations and/or kits thereof to a third party.

In other embodiments, a sales force may target a subset of healthcare providers that treat/prevent a condition herein. For example, a sales force may sell the compositions, formulations, or kits herein to cardiologists to treat/prevent cardiovascular conditions including heart attacks, strokes, transmural or non-transmural myocardial infarctions, acute myocardial infarctions, coronary artery disease, coronary heart disease, arrhythmia, sudden cardiac death, cerebrovascular accident, congestive heart failure, life-threatening dysrhythmia, cardiomyopathy, transient ischemic attacks, acute ischemic syndrome, and angina pectoralis, or a combination thereof.

In another embodiment, the sales force may sell compositions, formulations, or kits to general physicians for treatment or prevention of conditions, such as the microvascular disease of diabetes mellitus, acute bowel ischemia, bowel ischemia, kidney ischemia, intermittant claudication of skeletal muscle, migraine headaches, Raynaud's phenomenon, a hepatic injury, a pancreatic injury, and disseminated intravascular coagulation such as due to bowel ischemia, and shock, or a combination thereof.

In another embodiment, the sales force may sell compositions, formulation, or kits to surgeons in the case of patients undergoing a surgical procedure, including coronary artery bypass grafting (CABG) surgery, percutaneous transluminal coronary angioplasty (PTCA), laser angioplasty, cerebral angioplasty, an atherectomy, an intravascular stent procedure, carotid endarterectomy, valve replacement surgery, and organ transplantation surgery, small and large bowel resection, organ transplantation, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, and liposuction, or a combination thereof. In one embodiment, the sales force may sell to anesthesiologists for the purpose of administering a formulation from the kit in conjunction with an anesthetic during a surgery described herein.

All of the embodiments and examples herein are in no way intended to limit the scope of the instant invention. Further, it can be appreciated to one of ordinary skill in the art that many changes and modifications can be made thereto without departing from the spirit or scope of the appended claims, and such changes and modifications are contemplated within the scope of the instant invention.

All patents, publications, and patent applications mentioned in this specification are herein incorporated by reference to the same extent as if each individual patent, publication, or patent application was specifically and individually indicated to be incorporated by reference.

What is claimed is:

1. A buffered solution comprising AICA riboside, wherein said buffered solution has a pH of 6.5-7.5.

2. The solution as recited in claim 1 wherein said buffered solution comprises a buffer selected from the group consisting of Tris, phosphate, and histidine.

3. The solution as recited in claim 1 wherein said AICA riboside is sterilized.

4. The solution as recited in claim 1 wherein said AICA riboside is sterilized by lyophilization.

5. The solution as recited in claim 1 or 2 wherein said AICA riboside is sterilized by means other than lyophilization.

6. The solution as recited in claim 1 wherein said solution includes less than 1% impurity by weight.

7. A perfusate solution comprising the solution as recited in claim 1, wherein said perfusate solution further comprises an excipient.

8. The solution of claim 1 further comprising a second therapeutic agent.

9. A method of treating or preventing a condition in a patient comprising:
administering to the patient an effective amount of a solution as recited in claim 1.

10. The method of claim 9 wherein said condition is selected from the group consisting of a heart attack, a stroke, a transmural or non-transmural myocardial infarction, an acute myocardial infarction, coronary artery disease, coronary heart disease, an arrhythmia, sudden cardiac death, a cerebrovascular accident, congestive heart failure, a life-threatening dysrhythmia, cardiomyopathy, a transient ischemic attack, an acute ischemic syndrome, angina pectoralis, the microvascular disease of diabetes mellitus, acute bowel ischemia, kidney ischemia, intermittant claudication of skeletal muscle, migraine headaches, Raynaud's phenomenon, a hepatic injury, a pancreatic injury, and, disseminated intravascular coagulation such as due to bowel ischemia, shock, or a combination thereof.

11. The method as recited in claim 10 wherein said condition is death.

12. The method as recited in claim 10 wherein said condition is a myocardial infarction.

13. The method as recited in claim 10 wherein said condition is a heart attack.

14. The method as recited in claim 9 wherein the patient is undergoing surgery.

15. The method as recited in claim 14 wherein said surgery is a cardiac surgery.

16. The method as recited in claim 15 wherein said cardiac surgery is selected from the group consisting of coronary artery bypass grafting (CABG), percutaneous transluminal coronary angioplasty (PTCA), laser angioplasty, cerebral angioplasty, an atherectomy, an intravascular stent procedure, carotid endarterectomy, heart transplant, implantation of artificial heart devices and defibrillators, valve replacement or repair, and congenital surgery.

17. The method as recited in claim 14 wherein said surgery is a non-cardiac surgery.

18. The method as recited in claim 17 wherein said non-cardiac surgery is selected from the group consisting of non-heart organ transplant, small and large bowel resection, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, and liposuction, or a combination thereof.

19. The method as recited in claim 9 wherein said solution is administered percutaneously.

20. The method as recited in claim 9 wherein said solution is administered by i.v.

21. The method as recited in claim 9 wherein said effective amount is between 0.001 mg/kg/min to 20 mg/kg/min.

22. The method as recited in claim 21 wherein said solution is administered for at least 1 hour.

23. The method as recited in claim 21 wherein said solution is administered for about 7 hours.

24. The method as recited in claim 9 wherein said solution is administered perioperatively.

25. The method as recited in claim 9 wherein said solution is administered by a perfusate solution.

26. The method as recited in claim 9 wherein said solution is administered by i.v. and a perfusate solution.

27. A method for preventing death, stroke, or a myocardial infarction in a patient undergoing CABG surgery comprising administering to said patient perioperatively an effective amount of the solution as recited in claim 1.

28. A kit comprising:
a first container comprising the solution as recited in claim 1, and
instructions for use thereof in preventing a condition in a patient.

29. The kit of claim 23 wherein said condition is selected from the group consisting of a heart attack, a stroke, a transmural or non-transmural myocardial infarction, an acute myocardial infarction, coronary artery disease, coronary heart disease, an arrhythmia, sudden cardiac death, a cerebrovascular accident, congestive heart failure, a life- threatening dysrhythmia, cardiomyopathy, a transient ischemic attack, an acute ischemic syndrome, angina pectoralis, the microvascular disease of diabetes mellitus, acute bowel ischemia, kidney ischemia, intermittant claudication of skeletal muscle, migraine headaches, Raynaud's phenomenon, a hepatic injury, a pancreatic injury, and disseminated intravascular coagulation such as due to bowel ischemia, shock, or a combination thereof.

30. The kit as recited in claim 23 wherein said condition is death, a myocardial infarction, or a heart attack.

31. The kit as recited in claim 23 wherein the patient is undergoing surgery.

32. The kit as recited in claim 25 wherein said surgery is a cardiac surgery.

33. The kit as recited in claim 25 wherein said surgery is a non-cardiac surgery.

34. A lyophilized composition comprising AICA riboside and a buffer.

35. The lyophilized composition of claim 28 wherein said buffer is selected from the group consisting of a Tris buffer, a phosphate buffer, and a histidine buffer.

36. The lyophilized composition of claim 28 wherein said composition has a pH between 6.5 and 7.5.

37. A kit comprising:
a first container comprising the solution as recited in claim 28, and
instructions for reconstituting said composition; and
instructions for use thereof in preventing a condition in a patient.

38. The kit of claim 31 wherein said condition is selected from the group consisting of a heart attack, a stroke, a transmural or non-transmural myocardial infarction, an acute myocardial infarction, coronary artery disease, coronary heart disease, an arrhythmia, sudden cardiac death, a cerebrovascular accident, congestive heart failure, a life- threatening dysrhythmia, cardiomyopathy, a transient ischemic attack, an acute ischemic syndrome, angina pectoralis, the microvascular disease of diabetes mellitus, acute bowel ischemia, kidney ischemia, intermittant claudication of skeletal muscle, migraine headaches, Raynaud's phenomenon, a hepatic injury, a pancreatic injury, and disseminated intravascular coagulation such as due to bowel ischemia, shock, or a combination thereof.

39. The kit of claim 31 wherein said first container is a glass container.
